(19) United States
(12) Patent Application Publication
(10) Pub. No.: US 2007/0203216 A1
(43) Pub. Date: Aug. 30, 2007

(54) METHOD OF TREATING INFLAMMATORY DISEASES

(76) Inventors: Bjarke Ebert, Farum (DK); Timothy P. Bonnert, Much Hadham (GB); Peter Haynes Hutson, Landsdale, PA (US); Richard Anthony Rutter, Singapore (SG)

Correspondence Address: DARBY & DARBY P.C., P.O. BOX 770, Church Street Station, New York, NY, 10008-0770, US

(21) Appl. No.: 11/673,898

(22) Filed: Feb. 12, 2007

Related U.S. Application Data
(60) Provisional application No. 60/773,475, filed on Feb. 14, 2006.

(51) Int. Cl. A61K 31/4172; A61K 31/195

(52) U.S. Cl. 514/400; 514/561

(57) ABSTRACT

The present invention relates to the use of gaboxadol, or a combination of gaboxadol and one or more anti-inflammatory compounds, for the treatment of an inflammatory disease. The present invention further relates to a pharmaceutical composition comprising gaboxadol and one or more anti-inflammatory compounds. The present invention further relates to a method of treating a disease wherein one or more inflammatory markers are increased, comprising administering to a subject in need thereof a therapeutically effective amount of a compound that enhances GABA_{A}-ergic neurotransmission.

### FIELD OF THE INVENTION

The present invention relates to the use of gaboxadol, or a combination of gaboxadol and one or more anti-inflammatory compounds, for the treatment of an inflammatory disease. The present invention further relates to a pharmaceutical composition comprising gaboxadol and one or more anti-inflammatory compounds. The present invention further relates to a method of treating a disease wherein one or more inflammatory markers are increased, comprising administering to a subject in need thereof a therapeutically effective amount of a compound that enhances GABA_{A}-ergic neurotransmission.

### BACKGROUND OF THE INVENTION

Receptors for the major inhibitory neurotransmitter, gammaamino butyric acid (GABA), are divided into two main classes: GABA_{A} receptors, which are members of the ligand gated ion channel superfamily; and the GABA_{B} receptors, which are G-protein coupled receptors.

GABA_{A} receptors are formed as a pentameric assembly of different families of receptor subunits. The assembly, which in most receptors includes 2 α subunits, 2 β subunits and a γ or δ subunit, determines the pharmacology of the functional receptor. The binding site for benzodiazepines is located at the interface between the α and γ subunit, whereas the binding site for GABA and other GABA_{A} agonists is located at the interface between the α and β subunit.

GABA_{A} receptor assemblies, which do exist, include, amongst many others, α₁β₂γ₂, α₁β_{2/3}γ₂, α₃βγ_{2/3}, α₅β₃γ_{2/2}, α₆βγ₂ α₆βδ, α₄βδ and α₄β₂γ₂. Subtypes containing the α₁ subunit are present in most brain regions and may contribute to the functional action of a number of benzodiazepines.

In a number of clinical conditions, hypoactivity of the inhibitory GABA system has been hypothesised as the underlying mechanism of the pathology in question.

Gaboxadol (4,5,6,7-tetrahydroisoxazolo [5,4-c] pyridine-3-ol) (THIP) is described in EP Patent No. 0000338 and in EP Patent No. 0840601, and has previously shown great potential in the treatment of sleep disorders. Gaboxadol has the following general formula:

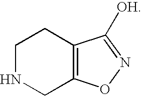

Gaboxadol may be prepared using methods that are well known in the art. For example, the following reaction scheme shows a full synthesis of gaboxadol from a known starting material, as disclosed in EP Patent No. 0000338:

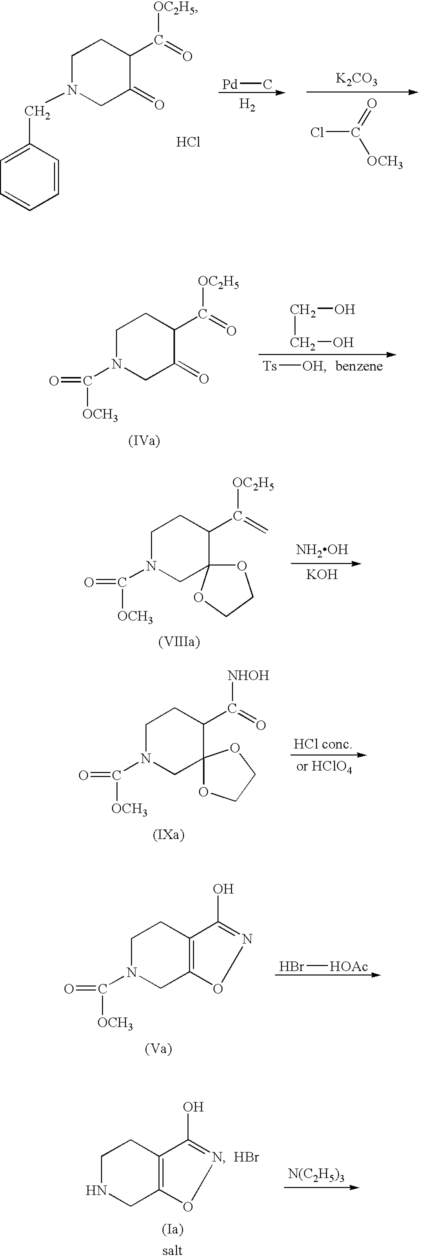

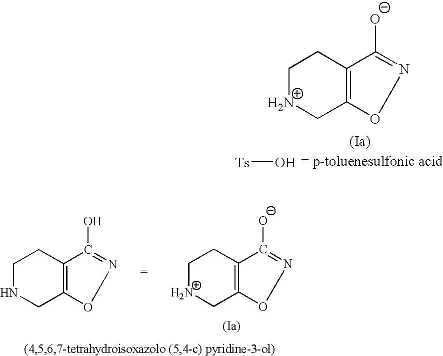

Inflammation comes as the immune system's first response to infection or irritation. Inflammatory diseases are typically characterized by one or more of the following symptoms: redness, swollen joints warm to the touch, joint pain, joint stiffness, and loss of joint function. Several treatments are currently available to decrease joint pain, swelling and inflammation, such as non-steroidal anti-inflammatory drugs (NSAIDs), corticosteroids (e.g., prednisone), anti-malarial agents (e.g., hydroxychloroquine), and acetaminophen.

U.S. Patent Application Publication No. 2005/0288371 discloses the use of gaboxadol for the preparation of medicaments useful for the treatment of neuropathic pain, fibromyalgia or rheumatoid arthritis. According to the publication, gaboxadol was found to inhibit the pain response in phase 2 of the formalin pain model.

There is still a need for improved anti-inflammatory formulations.

### SUMMARY OF THE INVENTION

The present inventors have discovered that gaboxadol significantly reduces the release of pro-inflammatory mediators and, therefore, is useful in the treatment of inflammatory diseases. Without being bound by any particular theory, the inventors theorize that gaboxadol activates GABA receptors on glia cells, which leads to their hyperpolarization and results in reduced release of pro-inflammatory mediators. This, in turn, reduces inflammation.

According to one embodiment of the invention, gaboxadol, or a pharmaceutically acceptable salt thereof, is used to effectively treat an inflammatory disease when administered either alone or in combination with one or more anti-inflammatory compounds, or a pharmaceutically acceptable salt thereof. In another embodiment, the inflammatory disease is not rheumatoid arthritis.

Another embodiment of the present invention is a method of treating an inflammatory disease in a subject in need thereof by administering to the subject a therapeutically effective amount of gaboxadol, or a pharmaceutically acceptable salt thereof, alone or in combination with one or more anti-inflammatory compounds, or a pharmaceutically acceptable salt thereof. In another embodiment, the amount of gaboxadol administered is effective to treat the inflammatory disease, but is less than a sleep-inducing amount. In one embodiment, the subject does not suffer from rheumatoid arthritis. In another embodiment, the subject does not suffer from rheumatoid arthritis, fibromyalgia, neuropathic pain, or any combination of the foregoing.

Yet another embodiment is a pharmaceutical composition comprising gaboxadol, or a pharmaceutically acceptable salt thereof, and one or more anti-inflammatory compounds, or a pharmaceutically acceptable salt thereof. In another embodiment, the pharmaceutical composition includes an anti-inflammatory effective amount of gaboxadol and the anti-inflammatory compound(s).

The present invention also relates to the use of gaboxadol, or a pharmaceutically acceptable salt thereof, alone or in combination with one or more anti-inflammatory compounds, or a pharmaceutically acceptable salt thereof, to decrease inflammation, increase immune response, increase resistance to bacterial or viral infection, increase the total lymphocyte count of a human (e.g., in a patient suffering from bacterial or viral infection), treat immunosuppressed patients, accelerate wound healing, treat delayed wound healing, accelerate recovery from surgery, accelerate the recovery of burn patients, or reduce hospitalization of burn patients, by administering a therapeutically effective amount of gaboxadol, alone or in combination with one or more anti-inflammatory compounds, to a subject in need thereof.

The present invention also relates to a method of treating a disease wherein one or more inflammatory markers are increased, comprising administering to a subject in need thereof a therapeutically effective amount of a compound that enhances GABA_{A}-ergic neurotransmission. In yet another embodiment the invention relates to a pharmaceutical composition comprising a compound that enhances GABA_{A}-ergic neurotransmission and one or more anti-inflammatory compounds, or a pharmaceutically acceptable salt thereof.

The present invention also relates to the use of the compound(s) of the invention for the manufacture of a pharmaceutical composition for treating a disease wherein one or more inflammatory markers are increased. In one embodiment the disease is an inflammatory disease. In another embodiment the inflammatory disease is not rheumatoid arthritis.

### DESCRIPTION OF THE INVENTION

### Definitions

As used herein, the term “inflammatory disease” refers to either an acute or chronic inflammatory condition, which can result from infections or non-infectious causes. Various infectious causes include meningitis, encephalitis, uveitis, colitis, tuberculosis, dermatitis, and adult respiratory distress syndrome. Non-infectious causes include trauma (burns, cuts, contusions, crush injuries), autoimmune diseases, and organ rejection episodes. Thus, in specific embodiments, an inflammatory condition results from a condition selected from the group that includes: atherosclerosis (arteriosclerosis); autoimmune conditions, such as multiple sclerosis, systemic lupus erythematosus, polymyalgia rheumatica (PMR), gouty arthritis, degenerative arthritis, tendonitis, bursitis, psoriasis, fibrosis, arthrosteitis, rheumatoid arthritis and other forms of inflammatory arthritis, Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroditis, Graves' disease, Goodpasture's disease, mixed connective tissue disease, sclerosing cholangitis, inflammatory bowel disease including Crohn's Disease (regional enteritis) and ulcerative colitis, pernicious anemia, inflammatory dermatoses; usual interstitial pneumonitis (UIP), asbestosis, silicosis, bronchiectasis, berylliosis, talcosis, all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa); inflammatory dermatoses not presumed to be autoimmune; chronic active hepatitis; delayed-type hypersensitivity reactions (e.g., poison ivy dermatitis); pneumonia or other respiratory tract inflammation due to any cause; Adult Respiratory Distress Syndrome (ARDS) from any etiology; encephalitis with inflammatory edema; immediate hypersensitivity reactions including, but not limited to, asthma, hayfever, cutaneous allergies, acute anaphylaxis; diseases involving acute deposition of immune complexes, including, but not limited to, rheumatic fever, acute and/or chronic glomerulonephritis due to any etiology, including specifically post-infectious (e.g., post-Streptococcal) glomerulonephritis, acute exacerbations of Systemic Lupus Erythematosus; pyelonephritis; cellulitis; cystitis; acute and/or chronic cholecystitis; and conditions producing transient ischemia anywhere along the gastrointestinal tract, bladder, heart, or other organ, especially those prone to rupture; sequelae of organ transplantation or tissue allograft, including allograft rejection in the acute time period following allogeneic organ or tissue transplantation and chronic host-versus-graft rejection. The term “inflammatory disease” also includes appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, tonsillitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, hepatitis, and necrotizing enterocolitis.

Any anti-inflammatory compound(s), or a pharmaceutically acceptable salt thereof, may be used in combination with gaboxadol for the treatment of an inflammatory disease, or for the treatment of inflammation and pain, according to the present invention. Suitable anti-inflammatory compounds include, but are not limited to: non-steroidal anti-inflammatory drugs (NSAIDs) (e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), anti-malarial agents (e.g., hydroxychloroquine), acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, and antibiotics.

As used herein, the term “subject” refers to any mammal. The subject, such as a human, to be treated with gaboxadol may in fact be any subject of the human population, male or female, which may be divided into children, adults, or elderly. Any one of these patient groups relates to an embodiment of the invention. In one embodiment, the subject is an elderly human. In one embodiment, the subject does not suffer from a sleep disorder or sleep condition.

As used herein, the term “therapeutically effective amount” refers to the amount/dose of a compound or pharmaceutical composition that is sufficient to produce an effective response (i.e., a biological or medical response of a tissue, system, animal or human sought by a researcher, veterinarian, medical doctor or other clinician) upon administration to a subject. The “therapeutically effective amount” will vary depending on inter alia the disease and its severity, and the age, weight, physical condition and responsiveness of the subject to be treated.

As used herein, the term “treating” refers to preventing or delaying the appearance of clinical symptoms of a disease or condition in a subject that may be afflicted with or predisposed to the disease or condition, but does not yet experience or display clinical or subclinical symptoms of the disease or condition. “Treating” also refers to inhibiting the disease or condition, i.e., arresting or reducing its development or at least one clinical or subclinical symptom thereof. “Treating” further refers to relieving the disease or condition, i.e., causing regression of the disease or condition or at least one of its clinical or subclinical symptoms. The benefit to a subject to be treated is either statistically significant or at least perceptible to the subject and/or the physician.

As used herein, the term “pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Throughout this description, “gaboxadol” is intended to include any form of the compound, such as the free base (zwitter ion), pharmaceutically acceptable salts, e.g., pharmaceutically acceptable acid addition salts, hydrates or solvates of the base or salt, as well as anhydrates, and also amorphous, or crystalline forms.

In a further embodiment, gaboxadol is selected from the zwitter ion, typically a hydrate thereof, although the anhydrate is also suitable. A suitable embodiment is the zwitter ion monohydrate.

In a further embodiment, gaboxadol is selected from an acid addition salt, typically a pharmaceutically acceptable acid addition salt. A suitable embodiment is an organic acid addition salt, such as any one of the maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. Another suitable embodiment is an inorganic acid addition salt, such as any one of the hydrochloric, hydrobromic, sulfuric, sulfamic, phosphoric or nitric acid addition salts.

In another embodiment, gaboxadol is in the form of the hydrochloric acid salt, the hydrobromic acid salt, or the zwitter ion monohydrate.

In a further embodiment, gaboxadol is crystalline, such as the crystalline hydrochloric acid salt, the crystalline hydrobromic acid salt, or the crystalline zwitter ion monohydrate.

The acid addition salts according to the invention may be obtained by treatment of gaboxadol with the acid in an inert solvent followed by precipitation, isolation and optionally re-crystallization by known methods and if desired micronization of the crystalline product by wet or dry milling or another convenient process, or preparation of particles from a solvent-emulsification process. Suitable methods are described in EP Patent No. 0000338, for example.

Precipitation of the salt is typically carried out in an inert solvent, e.g., an inert polar solvent such as an alcohol (e.g., ethanol, 2-propanol and n-propanol), but water or mixtures of water and inert solvent may also be used.

In another embodiment the compound that enhances GABA_{A}-ergic neurotransmission is selected from the group comprising GABA_{A} agonists, allosteric modulators of the GABA_{A} receptor complex and GABA_{A} uptake inhibitors. In one embodiment the compound that enhances GABA_{A}-ergic neurotransmission is selected from the group comprising gaboxadol, cyclopropylGABA, isoguvacine, muscimol, imidazole-4-acetic acid, gabapentin and tiagabine, or a pharmaceutically acceptable salt thereof. In yet another embodiment the inflammatory marker is selected from the group comprising Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin) and VEGF (Vascular Endothelial Cell Growth Factor).

In yet another embodiment the disease wherein one or more inflammatory markers are increased, is an inflammatory disease. In another embodiment the inflammatory disease is not rheumatoid arthritis.

In yet another embodiment the compound that enhances GABA_{A}-ergic neurotransmission is administered in combination with one or more anti-inflammatory compounds, or a pharmaceutically acceptable salt thereof. In yet another embodiment the pharmaceutical composition comprising a compound that enhances GABA_{A}-ergic neurotransmission and one or more anti-inflammatory compounds, said compound that enhances GABA_{A}-ergic neurotransmission is selected from the group comprising GABA_{A} agonists, allosteric modulators of the GABA_{A} receptor complex and GABA_{A} uptake inhibitors. In one embodiment the compound is selected from the group comprising gaboxadol, cyclopropylGABA, isoguvacine, muscimol, imidazole-4-acetic acid, gabapentin and tiagabine, or a pharmaceutically acceptable salt thereof. In yet another embodiment the pharmaceutical composition comprising a compound that enhances GABA_{A}-ergic neurotransmission and one or more anti-inflammatory compounds, the combined amount of the compound that enhances GABA_{A}-ergic neurotransmission and anti-inflammatory compound(s) is effective to treat an inflammatory disease.

### Formulations

The invention also provides the use as above wherein the medicament is for administration as a unit dose. In another embodiment of the invention, the unit dose is containing the active ingredient in an amount from about 10 μg/kg to 10 mg/kg body weight, in another embodiment from about 25 μg/day/kg to 1.0 mg/day/kg, in yet another embodiment from about 0.1 mg/day/kg to 1.0 mg/day/kg body weight. In another embodiment, the unit dose is containing the active ingredient in an amount from 0.1 mg/day/kg to 1.0 mg/day/kg body weight.

According to the invention, the compounds mentioned above may be used as the base of the compound or as a pharmaceutically acceptable acid addition salt thereof or as an anhydrate or hydrate of such salt. According to the invention, the compounds mentioned above or a pharmaceutically acceptable salt thereof may be administered in any suitable way e.g. orally or parenterally, and it may be presented in any suitable form for such administration, e.g. in the form of tablets, capsules, powders, syrups or solutions or dispersions for injection. In another embodiment, and in accordance with the purpose of the present invention, the compound of the invention is administered in the form of a solid pharmaceutical entity, suitably as a tablet or a capsule or in the form of a suspension, solution or dispersion for injection. The compound of the invention is most conveniently administered orally in unit dosage forms such as tablets or capsules, containing the active ingredient in an amount from about 10 μg/kg to 10 mg/kg body weight, for example 25 μg/day/kg to 1.0 mg/day/kg.

Gaboxadol may be administered as an oral dose form, such as a solid oral dose form, typically tablets or capsules, or as a liquid oral dose form. Gaboxadol may be administered in an immediate release dosage form or a controlled or sustained release dosage form. According to one embodiment, the dosage form provides controlled or sustained release of the gaboxadol in an amount less than a sleep-inducing amount. Gaboxadol may be conveniently administered orally in unit dosage forms, such as tablets or capsules, containing the active ingredient in an amount from about 0.1 to about 150 mg/day, from about 0.2 to about 100 mg/day, from about 0.5 to about 50 mg/day, from about 0.1 to about 50 mg/day, from about 1 to about 15 mg/day, or from about 2 to about 5 mg/day. Typically, the pharmaceutical composition comprises from about 0.5 mg to about 20 mg, such as about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg or about 20 mg of gaboxadol. The amount of gaboxadol is calculated based on the free base (zwitter ion) form.

In one embodiment, gaboxadol is administered once daily (for example, in the morning or afternoon) using doses of about 2.5 mg to about 20 mg. In another embodiment, gaboxadol is administered in a more prolonged and continuous release using non-sleep-inducing concentrations of gaboxadol—e.g., administration 2-3 times daily with low doses or a modified release formulation prepared using conventional methods known in the art, such that about 5 to about 50 mg of gaboxadol are administered to the subject per 24 hour period. In yet another embodiment, gaboxadol is administered in an amount that is less than a sleep-inducing amount.

According to the present invention, gaboxadol or a pharmaceutically acceptable salt thereof may be administered in any suitable way, e.g., orally or parenterally, and it may be presented in any suitable form for such administration, e.g., in the form of tablets, capsules, powders, syrups or solutions or dispersions for injection. In another embodiment, and in accordance with the purpose of the present invention, gaboxadol is administered in the form of a solid pharmaceutical entity, suitably as a tablet or a capsule or in the form of a suspension, solution or dispersion for injection. Additionally, gaboxadol may be administered with a pharmaceutically acceptable carrier, such as an adjuvant and/or diluent.

Methods for the preparation of solid or liquid pharmaceutical preparations are well known in the art. See, e.g., Remington: The Science and Practice of Pharmacy, 21st ed., Lippincott Williams & Wilkins (2005). Tablets may thus be prepared by mixing the active ingredients with an ordinary carrier, such as an adjuvant and/or diluent, and subsequently compressing the mixture in a tabletting machine. Non-limiting examples of adjuvants and/or diluents include: corn starch, lactose, talcum, magnesium stearate, gelatine, lactose, gums, and the like. Any other adjuvant or additive such as colourings, aroma, and preservatives may also be used provided that they are compatible with the active ingredients. The pharmaceutical compositions of the invention thus typically comprise an effective amount of gaboxadol and a pharmaceutically acceptable carrier.

A suitable formulation of gaboxadol is described in WO 02/094225. Without limiting the invention in any way, it is intended that any one of the aspects or embodiments of this patent application is suitable for the medicaments or pharmaceutical compositions described herein. For example, WO 02/094225 entitled “Granular Preparations of Gaboxadol” relates to a specific melt granulation, which is particularly useful for formulation of an acid addition salt, but the present invention is in no way limited to such a formulation.

Pharmacological Tests

The following tests were performed to evaluate the potential effect of gaboxadol on stress related biochemical changes, using the Chronic mild stress model in rats (Jayatissa M N, Bisgaard C, Tingstrom A, Papp M, Wiborg O. Hippocampal cytogenesis correlates to escitalopram-mediated recovery in a chronic mild stress rat model of depression. Neuropsychopharmacology. 2006 November; 31(11):2395-404). The biochemical changes induced in the animals studied in the stress models lead to increases in inflammatory mediators.

At the end of the present study, terminal serum samples were collected for marker analysis to evaluate the effects of stress with and without drug on the levels of about 60 blood markers. Previously published data suggests that chronic mild stress modulates the expression of a number of blood plasma proteins. Differential modulation of this response by gaboxadol and escitalopram may be indicative of different mechanisms of action and may be alternative predictors of clinical efficacy.

Experimental Procedure

Animals

Serum samples were withdrawn from the tail, without stunning, during the daytime (09:00-17:00) 24 hrs after the last drug injection. Blood was collected into BD vacutainers containing clot activator and gel for serum preparation, inverted 5 times and maintained on ice until centrifugation at 3000 rpm for 10 min at 4° C. Serum was decanted, placed on ice, and at the end of the day stored at −80° C. Animal treatment groups were as follows (see table 1):

TABLE 1
Animal sets.
ANIMAL SETS

| | | Drug Treatment |
|---|---|---|
| Treatment Code | Stress Conditioning | or Behavior |
| CMS-VEH | Chronic Mild Stress | Vehicle |
| CMS-ECT | Chronic Mild Stress | Escitalopram |
| CMS-GBX-5 mg/kg | Chronic Mild Stress | Gaboxadol |
| CMS-GBX-10 mg/kg | Chronic Mild Stress | Gaboxadol |
| CMS-RES | Chronic Mild Stress | Resistant |
| NS-VEH | Non-Stressed | Vehicle |
| NS-ECT | Non-Stressed | Escitalopram |
| NS-GBX | Non-Stressed | Gaboxadol |

Analysis

Subsequent analyses of Serum samples showed the following (see table 2):

TABLE 2
Median values of each tested factor for each animal treatment group.

| | | median values | | | | | | |
|---|---|---|---|---|---|---|---|---|
| | | 1st | | | | | | |
| | | study | | | | 2nd | | |
| | | | | GBX | | study | | |
| Blood parameter | | control | stress | 5 mg/kg | ESC 5 mg/kg | control | stress | GBX 10 mg/kg |
| Apo A1 (Apolipoprotein | ug/mL | 7.00 | 7.30 | 8.20 | 8.40 | 4.80 | 5.80 | 6.20 |
| A1) | | | | | | | | |
| Beta-2 Microglobulin | ug/mL | | | | | 45.00 | 59.00 | 38.00 |
| Calbindin | ng/mL | | | | | 0.40 | 0.12 | |
| Clusterin | ug/mL | | | | | 110.00 | 146.00 | 108.00 |
| CRP (C Reactive Protein) | ug/mL | 1235.00 | 1105.00 | 1140.00 | 1060.00 | 1130.00 | 1190.00 | 1030.00 |
| Cystatin-C | ng/mL | 1045.00 | 1440.00 | 1165.00 | | 717.00 | 956.00 | 696.00 |
| Eotaxin | pg/mL | 851.00 | 1210.00 | | 1300.00 | 1362.00 | 1656.00 | 1072.00 |
| Factor VII | ng/mL | | | | | 0.48 | 0.07 | 0.30 |
| FGF-9 (Fibroblast Growth | ng/mL | 0.38 | 0.95 | 0.59 | 0.90 | 1.70 | 2.40 | 1.70 |
| Factor-9) | | | | | | | | |
| GCP-2 (Granulocyte | ng/mL | 0.14 | 0.23 | 0.19 | 0.21 | 0.28 | 0.31 | 0.29 |
| Chemotactic Protein-2) | | | | | | | | |
| Growth Hormone | ng/mL | 1.66 | 5.67 | 7.67 | 1.38 | 12.00 | 23.00 | 3.10 |
| GST-alpha (Glutathione S- | ng/mL | | | | | 0.89 | 0.40 | |
| Transferase alpha) | | | | | | | | |
| GST-Mu | ng/mL | | | | | 2080.00 | 60.00 | 1190.00 |
| IgA (Immunoglobulin A) | ug/mL | 7.10 | 6.83 | 5.91 | 6.54 | 5.70 | 7.60 | 4.30 |
| IL-10 (Interleukin-10) | pg/mL | 278.00 | 228.00 | 249.00 | 317.00 | 360.00 | 411.00 | 362.00 |
| IL-11 (Interleukin-11) | pg/mL | 129.00 | 105.00 | 128.00 | 166.00 | 164.00 | 98.00 | |
| IL-18 (Interleukin-18) | ng/mL | 0.07 | 0.14 | 0.07 | | 0.53 | 0.85 | 0.43 |
| IL-1alpha (Interleukin- | pg/mL | 8.12 | 13.80 | 11.30 | 15.40 | | | |
| 1alpha) | | | | | | | | |
| IL-1beta (Interleukin-1beta) | ng/mL | | | | | 0.53 | 0.65 | 0.56 |
| IL-2 (Interleukin-2) | pg/mL | 22.30 | 38.80 | 31.30 | 38.80 | 33.00 | 46.00 | 22.00 |
| IL-4 (Interleukin-4) | pg/mL | 24.65 | 46.70 | 29.60 | 51.70 | | | |
| IL-5 (Interleukin-5) | ng/mL | | | | | 0.26 | 0.51 | 0.24 |
| IL-7 (Interleukin-7) | ng/mL | 0.11 | 0.22 | 0.16 | 0.22 | 0.04 | 0.04 | 0.06 |
| Insulin | ulU/mL | 10.90 | 12.20 | 8.88 | 11.30 | 9.40 | 3.10 | 14.50 |
| IP-10 (Inducible Protein-10) | pg/mL | 86.70 | 186.00 | 138.00 | 194.00 | 31.00 | 36.00 | 41.00 |
| Leptin | ng/mL | 3.24 | 3.17 | 2.59 | 2.62 | 0.94 | 0.27 | 1.35 |
| LIF (Leukemia Inhibitory | pg/mL | 36.60 | 70.50 | 53.90 | 67.20 | 49.00 | 93.00 | 67.00 |
| Factor) | | | | | | | | |
| Lymphotactin | pg/mL | 41.80 | 73.70 | 53.80 | 68.00 | 64.00 | 72.00 | 63.00 |
| MCP-1 (Monocyte | pg/mL | 1095.00 | 1095.00 | 1185.00 | 1500.00 | 1065.00 | 1025.00 | 1020.00 |
| Chemoattractant Protein-1) | | | | | | | | |
| MCP-3 (Monocyte | pg/mL | 566.00 | 657.00 | 655.00 | 885.00 | 653.00 | 703.00 | 659.00 |
| Chemoattractant Protein-3) | | | | | | | | |
| MCP-5 (Monocyte | pg/mL | 0.76 | 0.73 | 0.77 | 0.87 | 0.75 | 0.75 | 0.72 |
| Chemoattractant Protein-5) | | | | | | | | |
| M-CSF (Macrophage- | ng/mL | 813.00 | 875.00 | 865.00 | 941.00 | 1960.00 | 1610.00 | 1400.00 |
| Colony Stimulating Factor) | | | | | | | | |
| MDC (Macrophage-Derived | pg/mL | | | | | 0.13 | 0.26 | 0.12 |
| Chemokine) | | | | | | | | |
| MIP-1alpha (Macrophage | ng/mL | 116.00 | 209.00 | 173.00 | 244.00 | 120.00 | 107.00 | 127.00 |
| Inflammatory Protein- | | | | | | | | |
| 1alpha) | | | | | | | | |
| MIP-1beta (Macrophage | pg/mL | | | | | 0.01 | 0.02 | 0.01 |
| Inflammatory Protein-1beta) | | | | | | | | |
| MIP-1gamma (Macrophage | ng/mL | 6.42 | 13.10 | 7.95 | 11.50 | 16.00 | 15.00 | 19.00 |
| Inflammatory Protein- | | | | | | | | |
| 1gamma) | | | | | | | | |
| MIP-2 (Macrophage | pg/mL | 0.05 | 0.11 | 0.08 | 0.09 | 0.21 | 0.34 | 0.22 |
| Inflammatory Protein-2) | | | | | | | | |
| MIP-3beta (Macrophage | ng/mL | | | | | 21.75 | 13.70 | 29.70 |
| Inflammatory Protein-3beta) | | | | | | | | |
| MPO (Myeloperoxidase) | ng/mL | 89.40 | 53.80 | 20.80 | 54.60 | 610.00 | 39.85 | 533.00 |
| Myoglobin | ng/mL | | | | | 408.00 | 351.00 | 520.00 |
| NGAL (Lipocalin-2) | ng/mL | 0.21 | 0.12 | 0.17 | 0.22 | 0.10 | 0.11 | 0.15 |
| OSM (Oncostatin M) | ng/mL | | | | | 21.95 | 33.90 | 19.10 |
| Osteopontin | ng/mL | 188.00 | 391.00 | 396.00 | 310.00 | 356.00 | 509.00 | 255.00 |
| RANTES (Regulation Upon | pg/mL | | | | | 12.85 | 11.25 | 10.60 |
| Activation, Normal T-Cell | | | | | | | | |
| Expressed and Secreted) | | | | | | | | |
| SAP (Serum Amyloid P) | ug/mL | 135.00 | 173.00 | 133.00 | 178.00 | 241.00 | 198.00 | 403.00 |
| SCF (Stem Cell Factor) | pg/mL | 12.55 | 15.30 | 14.85 | 16.95 | 39.20 | 50.80 | 11.30 |
| SGOT (Serum Glutamic- | ug/mL | 0.16 | 0.27 | 0.21 | 0.31 | 6.16 | 6.14 | 8.53 |
| Oxaloacetic Transaminase) | | | | | | | | |
| TIMP-1 (Tissue Inhibitor of | ng/mL | 0.09 | 0.28 | 0.16 | 0.22 | 0.37 | 0.77 | 0.24 |
| Metalloproteinase Type-1) | | | | | | | | |
| Tissue Factor | ng/mL | 0.08 | 0.13 | 0.10 | 0.15 | 0.06 | 0.03 | 0.06 |
| TNF-alpha (Tumor Necrosis | ng/mL | | | | | 2.55 | 4.14 | 4.45 |
| Factor-alpha) | | | | | | | | |
| TPO (Thrombopoietin) | ng/mL | 134.00 | 142.00 | 147.00 | 142.00 | 130.00 | 167.00 | 119.00 |
| VCAM-1 (Vascular Cell | ng/mL | 299.00 | 381.00 | 361.00 | 409.00 | 330.00 | 311.00 | 353.00 |
| Adhesion Molecule-1) | | | | | | | | |
| VEGF (Vascular Endothelial | pg/mL | 55.00 | 89.00 | 81.00 | 106.00 | 69.00 | 243.00 | 78.00 |
| Cell Growth Factor) | | | | | | | | |
| vWF (von Willebrand | ng/mL | | | | | | | |
| Factor) | | | | | | | | |

Data

Numerical values of the concentration of each factor for each animal were collected.

### RESULTS AND CONCLUSION

The above-described pharmacological testing showed that chronic mild stress significantly alters a number of serum protein markers compared to non-stressed controls. Rats that did not show a behavioral response following CMS treatment (CMS-RES group) did not show a significant upregulation of these serum protein markers compared to non-stressed controls. The present inventors found that treatment with gaboxadol significantly reversed the stress-induced alterations in serum markers toward levels found in non-stressed controls.

Thus, from the foregoing testing and results, the inventors discovered that chronic mild stress significantly alters the expression of a set of serum marker proteins, and that partial or full reversal of this effect with gaboxadol (but not with escitalopram) suggests that gaboxadol affects stress-related biochemical changes by reducing inflammatory mediators. Thus gaboxadol, dose dependently is able to reverse or partially reverse changes at most in inflammatory parameters induced by chronic mild stress. In contrast, escitalopram was inactive at most of these changes. Thus, gaboxadol can be used to effectively treat inflammation and inflammatory diseases.

All non-patent references, patents, and patent applications cited and discussed in this specification are incorporated herein by reference in their entirety and to the same extent as if each was individually incorporated by reference.

1. A method of treating a disease wherein one or more inflammatory markers are increased, comprising administering to a subject in need thereof a therapeutically effective amount of a compound that enhances GABA_{A}-ergic neurotransmission.

2. The method of claim 1, wherein the compound is selected from the group comprising GABA_{A} agonists, allosteric modulators of the GABA_{A} receptor complex and GABA_{A} uptake inhibitors.

3. The method of claim 1, wherein the compound is selected from the group comprising gaboxadol, cyclopropylGABA, isoguvacine, muscimol, imidazole-4-acetic acid, gabapentin, tiagabine, and pharmaceutically acceptable salts thereof.

4. The method of claim 1, wherein the inflammatory marker is selected from the group comprising Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), IL-10 (Interleukin-10), IL-1beta (Interleukin-1beta), IL-2 (Interleukin-2), IL-4 (Interleukin-4), IL-5 (Interleukin-5), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin) and VEGF (Vascular Endothelial Cell Growth Factor).

5. The method of claim 1, wherein the disease is an inflammatory disease.

6. The method of claim 5, wherein the inflammatory disease is not rheumatoid arthritis.

7. The method of claim 5, wherein the inflammatory disease is selected from angitis, chronic bronchitis, pancreatitis, osteomylitis, glomerulonephritis, optic neuritis, temporal arteritis, encephalitis, meningitis, transverse myelitis, dermatomyositis, polymyositis, necrotizing fascilitis, hepatitis, and necrotizing enterocolitis.

8. The method of claim 1, wherein the compound is administered in combination with one or more anti-inflammatory compounds, or a pharmaceutically acceptable salt thereof.

9. The method of claim 8, wherein the anti-inflammatory compounds are selected from NSAIDs, corticosteroids, acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, and antibiotics.

10. The method of claim 1, wherein the subject is a human.

11. A pharmaceutical composition comprising a compound that enhances GABA_{A}-ergic neurotransmission and one or more anti-inflammatory compounds, or a pharmaceutically acceptable salt thereof.

12. The pharmaceutical composition of claim 11, wherein the anti-inflammatory compounds are selected from NSAIDs, corticosteroids, acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, and antibiotics.

13. The pharmaceutical composition of 11, wherein the compound is selected from the group comprising gaboxadol, cyclopropylGABA, isoguvacine, muscimol, imidazole-4-acetic acid, gabapentin, tiagabine, and pharmaceutically acceptable salts thereof.

14. The pharmaceutical composition of any of the claims 11, wherein the combined amount of the compound that enhances GABA_{A}-ergic neurotransmission and anti-inflammatory compound(s) is effective to treat an inflammatory disease.

15. A method of treating an inflammatory disease, comprising administering to a subject in need thereof a therapeutically effective amount of gaboxadol, or a pharmaceutically acceptable salt thereof, provided that the inflammatory disease is not rheumatoid arthritis.

16. The method of claim 15, wherein the inflammatory disease is selected from angitis, chronic bronchitis, pancreatitis, osteomylitis, glomerulonephritis, optic neuritis, temporal arteritis, encephalitis, meningitis, transverse myelitis, dermatomyositis, polymyositis, necrotizing fascilitis, hepatitis, and necrotizing enterocolitis.

17. The method of claim 15, wherein the gaboxadol is administered in combination with one or more anti-inflammatory compounds, or a pharmaceutically acceptable salt thereof.

18. The method of claim 17, wherein the anti-inflammatory compounds are selected from NSAIDs, corticosteroids, acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, and antibiotics.

19. The method of claim 15, wherein the gaboxadol is administered with a pharmaceutically acceptable carrier.

20. The method of claim 15, wherein the therapeutically effective amount is from about 0.1 mg/day to about 50 mg/day.

21. The method of claim 20, wherein the amount is less than a sleep-inducing amount.

22. The method of claim 15, wherein gaboxadol is administered in the morning.

23. The method of claim 15, wherein the subject is a human.

24. The method of claim 15, wherein the subject does not suffer from a sleep disorder or sleep condition.

25. A pharmaceutical composition comprising gaboxadol, or a pharmaceutically acceptable salt thereof, and one or more anti-inflammatory compounds, or a pharmaceutically acceptable salt thereof.

26. The pharmaceutical composition of claim 25, wherein the anti-inflammatory compounds are selected from NSAIDs, corticosteroids, acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, and antibiotics.

27. The pharmaceutical composition of claim 25, wherein the combined amount of gaboxadol and anti-inflammatory compound(s) is effective to treat an inflammatory disease.

28. The pharmaceutical composition of claim 25, wherein the combined amount of gaboxadol and anti-inflammatory compound(s) is less than a sleep-inducing amount.
